(19) United States
(12) Patent Application Publication
(10) Pub. No.: US 2021/0402012 A1
(43) Pub. Date: Dec. 30, 2021

(54) PARTICLE COMPRISING LANTHANIDE HYDROXIDE

(71) Applicant: QUIREM Medical B.V., Deventer (NL)

(72) Inventors: Johannes Franciscus Wilhelmus NIJSEN, Ugchelen (NL); Alexandra GIL ARRANJA, Reeuwijk (NL)

(73) Assignee: QUIREM Medical B.V., Deventer (NL)

(21) Appl. No.: 17/292,910

(22) PCT Filed: Dec. 16, 2019
(86) PCT No.: PCT/NL2019/050842

§ 371 Date: May 11, 2021

(30) Foreign Application Priority Data
Dec. 14, 2018 (EP) 18212617.7

(51) Int. Cl. A61K 49/18; C01F 17/229; C01F 17/206; A61K 33/244; A61B 6/03

(52) U.S. Cl. CPC A61K 49/1818; G01R 33/5601; C01F 17/206; A61K 49/1806; A61K 33/244; A61B 6/032; A61B 6/037; C01P 2004/03; C01P 2002/72; C01P 2002/82; C01P 2002/88; C01P 2004/32; C01P 2004/64; C01P 2004/62; C01P 2004/61; C01P 2006/42; C01F 17/229

(57) ABSTRACT

The disclosure is directed to a spherical particle comprising lanthanide hydroxide, a method of preparing the particle, the particle for use in medical applications, a suspension, a composition, a method of obtaining a scanning image, and the particle for use in the treatment of a subject.

The invention is directed to a spherical particle comprising lanthanide hydroxide, a method of preparing the particle, the particle for use in medical applications, a suspension, a composition, a method of obtaining a scanning image, and the particle for use in the treatment of a subject.

The invention relates to the use of a particle according to the invention in medical applications, such as the treatment, in particular by radiotherapy, of various forms of cancers and tumours.

Lanthanides, particularly holmium and yttrium, can be used in the treatment, in particular by radiotherapy, of various forms of cancers and tumours, such as those which can be found in the liver, head and neck, kidney, lung and the brain. Upon neutron irradiation holmium-165 (¹⁶⁵Ho) and yttrium-89 (⁸⁹Y) are converted to the radioactive isotopes ¹⁶⁶Ho and ⁹⁰Y, respectively, both of which are beta(β)-radiation emitters, and ¹⁶⁶Ho being a gamma(γ)-emitter as well. Consequently, both lanthanides can be used in nuclear imaging and radioablation. Lee et al., Eur. J. Nucl. Med. 2002, 29(2), 221-230 has shown that radioactive holmium can be effective in the radioablation treatment of malignant melanoma in a rat.

Further, it is known in the art that holmium can be visualised by computer tomography and magnetic resonance imaging (MRI) due to its high attenuation coefficient and paramagnetic properties, as described for instance by Bult et al., Pharm. Res. 2009, 26(6), 1371-1378.

Various attempts have been made to locally administer radionuclides, such as radioactive isotopes of lanthanides, particularly holmium, as a treatment for cancer. The main goal of these radionuclide therapies is to locally deliver tumouricidal doses of radiation to the tumours leaving healthy tissue unharmed.

McLaren et al., Eur. J. Nucl. Med. 1990, 16, 627-632 describes the use of ¹⁶⁵dysprosium hydroxide macroaggregates in animal studies relating to radiation synovectomy of certain forms of arthritis.

Huang et. al., New J. Chem. 2012, 36, 1335-1338 describes the synthesis and use of gadolinium hydroxide nanorods for magnetic resonance imaging.

WO-A-2013/096776 describes radioactive compositions used for treating bone cancer.

U.S. Pat. No. 4,752,464 discloses a radioactive composition for the treatment of arthritis comprising a ferric hydroxide or aluminium hydroxide aggregate suspension wherein radionuclide ¹⁶⁶holmium is entrapped.

WO-A-2009/011589 describes holmium acetylacetonate (Ho-acac) microspheres, the preparation thereof, and the use of the microspheres. The microspheres comprise high lanthanide metal content, complexed with a number of organic molecules, e.g. acetylacetonate, and no binder or only very small amounts of binder, such as poly(L-lactic acid). WO-A-2009/011589 shows that the reduction of binder material does not lead to a disintegration of the microspheres. These microspheres, comprising more than 20 wt. % of lanthanide metal, display a shorter neutron activation time and higher specific activity. Nevertheless, it would be desirable to design microspheres comprising compounds which are naturally occurring in the body, so that, when applied to a patient, possible toxic effects of the microspheres are minimised.

WO-A-2012/060707 describes holmium phosphate (HoPO₄) microspheres, the preparation thereof, and the use of the microspheres. These microspheres comprise a naturally occurring compound, i.e. phosphate, complexed with a lanthanide metal. However, it would be desirable to obtain a microsphere with an increased weight percentage of lanthanide metal, in order to lower the required amount of microspheres to be inserted into a body.

It is an objective of the invention to provide particles comprising lanthanide hydroxide, such as holmium hydroxide, for use in medical applications, in particular with respect to improving the stability of the particle in a liquid, such as an aqueous solution or a biological fluid, especially under neutral and acidic conditions.

Yet a further objective of the invention is to provide a method with which particles of the invention are prepared having a narrow distribution size.

Yet a further objective of the invention is to provide a particle that has a higher lanthanide content, in particular comprising holmium, in order to achieve higher specific activities.

Yet a further objective of the invention is to provide a particle that exhibits increased properties, e.g. stability to neutron activation and gamma irradiation.

Yet a further objective of the invention is to provide a particle that is stable in administration fluid after neutron activation, such as saline solution.

Yet a further objective of the invention is to provide a particle that is stable in human blood and implants.

The inventors found that one or more of these objectives can, at least in part, be met by providing a particle comprising lanthanide hydroxide.

Accordingly, in a first aspect of the invention there is provided a spherical particle comprising lanthanide hydroxide.

In a further aspect of the invention, there is provided a method of preparing the particle as described herein, comprising:
i) adding at least one metal particle to a salt solution to form a mixture;
ii) stirring the mixture to form the particle;
iii) recovering from at least part of the mixture of ii) the particle.

In yet a further aspect of the invention, there is provided a particle as described herein for use in medical applications.

In yet a further aspect of the invention, there is provided a suspension comprising the particle as described herein, the suspension being a therapeutic suspension, diagnostic suspension or a scanning suspension, such as a magnetic resonance imaging scanning suspension or a nuclear scanning suspension.

In yet a further aspect of the invention, there is provided a composition comprising the particle as described herein, or the suspension as described herein, wherein the particle or the particle present in the suspension further comprises a pharmaceutically acceptable carrier, diluent and/or excipient.

In yet a further aspect of the invention, there is provided a method of obtaining a scanning image, comprising:
i) administering to a human, humanoid, or nonhuman the suspension of the invention, and subsequently
ii) generating a scanning image of the human, humanoid, or nonhuman.

In yet a further aspect of the invention, there is provided the particle as described herein for use in the treatment of a subject, comprising:
i) administering to the subject a diagnostic composition or scanning composition, comprising the particle as described herein, the suspension as described herein, or the composition as described herein, wherein the particle is capable of at least in part disturbing a magnetic field;
ii) obtaining a scanning image of the subject;
iii) determining the distribution of the particle within the subject;
iv) administering to the subject a therapeutic composition comprising the particle as described herein, the suspension as described herein, or the composition as described herein, wherein the particle in the therapeutic composition has a higher amount of activity per particle than the particle in the diagnostic composition or scanning composition.

In yet a further aspect of the invention, there is provided a the particle as described herein capable of at least in part disturbing a magnetic field for use in the treatment of a tumour in a subject, wherein the dosage of the particle is derived from a scanning image obtained with a scanning suspension, such as the suspension as described herein, comprising particles capable of at least in part disturbing a magnetic field with the same chemical structure as the particle, based on the distribution of the particles of the scanning suspension with the same chemical structure within the subject, and wherein the particle for use in the treatment of the tumour exhibits a higher amount of radioactivity per particle than the particles used for obtaining the scanning image.

When referring to a noun (e.g. a particle, a metal complex, a solvent, etc.) in the singular, the plural is meant to be included, or it follows from the context that it should refer to the singular only.

The term “cancer”, as used herein, refers to a malignancy, such as a malignant tumour, which is typically a solid mass of tissue that is present (e.g. in an organ or the lymph system) in a subject, e.g. the human or animal body (i.e. human, humanoid or nonhuman body). The terms “cancer” and “tumour” are used interchangeably herein.

The terms “human”, “humanoid” and “nonhuman” as used herein, are meant to include all animals, including humans.

The term “subject” as used herein is meant to include the human and animal body, and the terms “individual” and “patient”.

The term “individual” as used herein is meant to include any human, humanoid or nonhuman entity.

The terms “treatment” and “treating” as used herein are not meant to be limited to curing. Treating is meant to also include alleviating at least one symptom of a disease, removing at least one symptom of a disease, lessen at least one symptom of a disease, and/or delaying the course of a disease. The term “treatment” as used herein is also meant to include methods of therapy and diagnosis.

The term “room temperature” as used herein is defined as the average indoor temperature to the geographical region where the invention is applied. In general, the room temperature is defined as a temperature of between about 18-25° C.

The term “Lewis base” as used herein is meant to refer to any chemical species, such as atomic and molecular species, where the highest occupied molecular orbital is highly localised. In other words, the Lewis base is a species that is capable of donating an electron pair, in particular to an electron acceptor (Lewis acid) to form a Lewis adduct, or complex. The bond formed in the Lewis acid-base reaction may be considered a non-permanent bond called a coordination covalent bond. The Lewis base can be regarded as a ligand when bonded to a metal or metalloid. The Lewis base can be solid or fluid, e.g. liquid, at room temperature. The Lewis base present in the particle as described herein is in the solid state.

As used herein, the term “ligand” refers to an atomic or molecular or ionic species that is bound in the vicinity of a metal or metalloid of a complex, such as a coordination complex. Since such a ligand can form a coordinate bond by providing a noncovalent electron pair to a metal or metalloid, it is essential to have a noncovalent electron pair so as to act as a ligand. According to the invention, the ligand is preferably characterised by being oxygenated and/or nitrogenous, whereto the oxygen and/or nitrogen acts as a donor atom that forms a coordinate bond by providing a noncovalent electron pair to a metal or metalloid.

As used herein, the term “monodentate” refers to a chemical species having one coordinate bond that can be formed with a metal or metalloid. The term “chelating ligand” as used herein refers to a ligand as described above having at least two coordinate bonds that can be simultaneously, though not necessarily, formed with a metal or metalloid.

One class of Lewis bases is neutral Lewis bases. The term “neutral” in “neutral Lewis base” as used herein is meant to refer to the non-ionic character of the Lewis base. Neutral Lewis bases are uncharged Lewis bases with non-bonded electrons that can be provided to an electron acceptor that is not in its ionic state. Several examples of neutral Lewis bases include, but are not limited to, water, ammonia, primary amines, such as ethylene diamine, secondary amines, tertiary amines, alcohols, ketones, such as β-dicarbonyl species exhibiting the keto-enol tautomerism (e.g. acetyl acetone), aldehydes, carboxylic acids, hydroxyl acids, thiols, and phosphines.

Another class of Lewis bases comprises Lewis bases that have an ionic character, and are charged. Such Lewis bases include, but are not limited to, hydride, oxide, hydroxide, alkoxides, carboxylates, such as oxalate, carbonate, nitrate, phosphate, sulphate, halides, thiolates, and acetyl acetonates.

In accordance with the invention, a particle is provided, in particular comprising holmium, with improved properties over known materials for use in medical applications, in particular with respect to imaging, neutron activation and treating cancer.

The invention provides a spherical particle comprising lanthanide hydroxide.

The shape and the dimensions of the particle of the invention may depend on the application of the particle. There are many descriptive terms that can be applied to the particle shape. Several shape classifications include, cubic, cylindrical, discoidal, ellipsoidal, equant, irregular, polygon, polyhedron, round, spherical, square, tabular, and triangular. In particular, the shape of the particle according to the invention may be classified as round. The shape of the particle of the invention is spherical. The disclosure further provides particles being spherical, rounded polyhedron, rounded polygon, such as poker chip, corn, pill, rounded cylinder, such as capsule, faceted. Preferably these particles are spherical, cylindrical, ellipsoidal or discoidal. More preferably, these particles are spherical particles. When compared to irregular particle shapes, the flow property of a spherical, cylindrical, ellipsoidal or discoidal particle in administration fluid(s) is improved. Ellipsoidal or cylindrical particle shapes may have a further advantage, e.g. in cell internalisation. The particle of the invention has a spherical shape such that its delivery to target sites is advantageous. The spherical particle experiences less flow resistance when administered as described herein. In addition, the particle typically has improved attrition resistance because of its shape.

The particle of the invention may have a certain sphericity and/or roundness. Sphericity is a measure of the degree to which a particle approximates the shape of a sphere-like object, and is independent of its size. Roundness is the measure of the sharpness of a particles edges and corners. Both sphericity and roundness are relative ratios and, therefore, dimensionless numbers. Sphericity and roundness may be determined based on Wadell's definitions, i.e. Wadell's sphericity and roundness indices, and/or by scanning electron microscopy. The sphericity of a particle may be determined by measuring the three linear dimensions of the particle (i.e., longest, intermediate and shortest diameters) and, for example, by using Zingg's diagram (1935). Wadell's sphericity of a particle is defined as follows:

$$Ψ = \frac{{( 36 π {V}^{2} )}^{\frac{1}{3}}}{S} ,$$

wherein Ψ is the sphericity, V is the volume of the particle, and S is the surface area of the particle. Roundness may be estimated by visually comparing grans of unknown roundness with standard images of grains of known roundness, for example, by using the method of Powers (1953). According to Wadell's definition, roundness is defined as follows:

$$R = \frac{\frac{1}{n} {Σ}_{i = 1}^{n} {r}_{i}}{{r}_{max}} ,$$

wherein R is the roundness, n is the number of corners, rᵢ is the radius of the i-th corner curvature, and rₘₐₓ is the radius of the maximum inscribed circle.

Alternatively, simplified parameters and/or visual charts may be used, such as methods that use three-dimensional imaging devices.

The particle as described herein may have a sphericity of 1.00 or less, and 0.50 or more, such as 0.60 or more, 0.75 or more, 0.85 or more, 0.90 or more, or 0.95 or more. In particular, the sphericity of the particle is 1.00 or less, and 0.85 or more, such as 0.87 or more, or 0.89 or more. Preferably, the sphericity is 1.00 or less, and 0.90 or more, 0.91 or more, 0.92 or more, 0.93 or more, 0.94 or more, or 0.95 or more. More preferably, the sphericity of the particle is 0.95-1.00. Even more preferably, the particle has a sphericity of 0.97-1.00. Most preferably, the sphericity is about 1.00, which is the upper limit. A particle with a sphericity of 1.00 represents a perfectly spherical particle.

The particle as described herein may have a roundness of 1.00 or less, and 0.50 or more, such as 0.60 or more, 0.75 or more, 0.85 or more, 0.90 or more, or 0.95 or more. In particular, the roundness of the particle is 1.00 or less, and 0.85 or more, such as 0.87 or more, or 0.89 or more. Preferably, the roundness is 1.00 or less, and 0.90 or more, 0.91 or more, 0.92 or more, 0.93 or more, 0.94 or more, or 0.95 or more. More preferably, the roundness of the particle is 0.95-1.00. Even more preferably, the particle has a roundness of 0.97-1.00. Most preferably, the roundness is about 1.00, which is the upper limit. A particle with a roundness of 1.00 represents a perfectly round particle.

The particle comprises at least lanthanide hydroxide. The amount of lanthanide hydroxide in the particle may be 0.1% or more, such as 0.5% or more, and 1% or more, based on the total weight of the particle. In particular, the lanthanide hydroxide content may be 100% or less and 10% or more, such as 20% or more, 30% or more, 40% or more, 50% or more, 65% or more, 75% or more, 80% or more, 85% or more, 90% or more, and 95% or more by total weight of the particle. Increased amounts of lanthanide hydroxide result in faster neutron activation (e.g. three times faster than with particles known from the prior art, such as poly(L-lactic acid) microspheres). Preferably, the amount of lanthanide hydroxide in the particle is 80-100% by total weight of the particle. Even more preferably, 100 wt. %. High amounts of lanthanide hydroxide, and possible other present metal complexes, result in more activity to be achieved within a neutron activation time. Consequently, the specific activity will increase as well, resulting in more activity and thus dose in a medical application. In addition, elevated activity levels due to high amounts of lanthanide hydroxide, and possible other present metal complexes contribute to a lowered amount of particles required which can be beneficial during for example radioembolisation or intratumoural injection. For example, in radioembolisation, too much particles will result in backflow and filling the normal healthy liver tissue, whereas for intratumoural injection there is only limited space, because the particles are injected interstitial (between the cells in tissue). The elevated activity as a result of high amounts of lanthanide hydroxide in the particle can also be used to overcome a longer transport time. When the lanthanide hydroxide content is low, the density of activity exhibited by the neutron activated particle is low. As a consequence thereof, a higher dose of neutron-activated particles is required to achieve the same effect as when using neutron-activated particles with a high lanthanide hydroxide content.

The particle comprises metal. In particular the metal may be lanthanide metal and/or transition metal. Preferably, the particle comprises lanthanide metal, scandium and/or yttrium. In the case the particle only comprises lanthanide hydroxide, the amount of metal in the particle may be 90% or less, and 0.1% or more, such as 0.5% or more, and 1% or more, based on the total weight of the particle. In particular, the metal content may be 90% or less, and 5% or more, 10% or more, 15% or more, 20% or more, 25% or more, 30% or more, 40% or more, 45% or more, 50% or more, 55% or more, 60% or more, 65% or more, 70% or more, 72.5% or more, 74% or more, 75% or more, 76% or more, 77% or more, 78% or more, 79% or more, 80% or more, 85% or more, 86% or more, or 87% or more, based on the total weight of the particle. Preferably, the amount of metal in the particle is 90% or less, and 46% or more, such as 63% or more, and 65% or more by total weight of the particle. More preferably, the amount of metal in the particle is 90% or less, and 74% or more, such as 75% or more, 76% or more, 77% or more, and 78% or more. Even more preferably, 90% or less, and 87 wt. % or more, such as 87.1% or more, 87.3% or more, 87.5% or more, 87.7% or more, and 88 wt. % or more. The amount of metal in the particle is controlled by difference between the atomic mass of the metal and the atomic mass or molecular weight of other species present. A high metal content will give a better scanning possibility, e.g. MRI, and for example even brings CT imaging of radioembolisation in reach. The particle comprising a minimum metal amount will still be usable for intratumoural CT (Computed Tomography) imaging. The above-mentioned advantages and disadvantages to the amounts of lanthanide hydroxide may also apply to the amount of atomic oxygen in the particle. For example, when the particle comprises scandium hydroxide, yttrium hydroxide, samarium hydroxide, gadolinium hydroxide, dysprosium hydroxide, holmium hydroxide, ytterbium hydroxide, or lutetium hydroxide, the atomic oxygen content may be about 46.9 wt. %, 63.5 wt. %, 74.7 wt. %, 75.5 wt. %, 76.1 wt. %, 76.4 wt. %, 77.2 wt. %, or 77.4 wt. %, respectively, based on the total weight of the particle.

The lanthanide hydroxide as part of the particle of the invention may comprise one or more metals selected from transition metals and/or lanthanide metals. In particular, the particle of the invention comprises one or more metals selected from the group consisting of scandium, yttrium, lanthanum, cerium, praseodymium, neodymium, promethium, samarium, europium, gadolinium, terbium, dysprosium, holmium, erbium, thulium, ytterbium and lutetium. Preferably, the metal hydroxide complex comprises one or more selected from the group consisting of scandium, yttrium, samarium, gadolinium, dysprosium, holmium, lutetium and ytterbium. More preferably, the lanthanide hydroxide comprises yttrium, dysprosium, holmium and/or lutetium. Even more preferably, the lanthanide hydroxide is holmium hydroxide, or dysprosium hydroxide.

In a specific embodiment, the metal comprises at least partially a radioactive isotope of above metal(s). The radioactive isotope of the metal may be generated by numerous methods, a non-exhaustive list includes neutron irradiation, laser pulse generation, laser-plasma interaction, cyclotron and using other sources of neutrons. For example, upon neutron irradiation ¹⁶⁵Ho is converted to ¹⁶⁶Ho. The particle of the invention may suitably be a radioactive particle. Preferably, however, the particle is initially non-radioactive, which has the advantage in that it avoids personnel being exposed to radiation and the need for specially equipped facilities, such as hot cells and transport facilities (i.e. prior to use in a medical application).

In an embodiment, the particle according to the invention comprises lanthanide hydroxide, such as dysprosium hydroxide or holmium hydroxide. In the case the lanthanide hydroxide comprises one or more metals as mentioned above, the obtainable particle comprises a relatively high amount of metal by total weight of the particle. Consequently, the particle comprising the high amount of metal has a higher specific activity when compared to known particles, e.g. holmium phosphate microspheres.

The particle according to the invention exhibits improved stability to neutron activation. Based on the current experimental results it is expected that the particle easily survives prolonged irradiation times (e.g. 10 hours) in high neutron fluxes (e.g. 4.1×10¹⁷ m⁻²s⁻¹).

The particle according to the invention has an atomic oxygen content. The atomic oxygen content of the particle may be 60% or less, and 1% or more, such as 5% or more, 7% or more, and 10% or more, based on the total weight of the particle. In particular, the atomic oxygen content of the particle may be 60% or less, and 10% or more, 11% or more, 12% or more, 12.5% or more, 13% or more, 13.5% or more, 15% or more, 17.5% or more, 20% or more, 21% or more, 22% or more, 22.5% or more, 23% or more, 23.5% or more, 25% or more, 30% or more, 31% or more, 32% or more 33% or more, 34% or more, 34.5% or more, 40% or more, 45% or more, or 50% more by total weight of the particle. Preferably, the atomic oxygen content in the particle is 10% or more, and 50% or less, 34.8% or less, 34.3% or less, 23.8% or less, 23.0% or less, 22.5% or less, 22.2% or less, 21.4 or less, 21.3% or less, 21.2% or less, 13.8% or less, 13.2% or less, 12.9% or less, 12.7% or less, 12.2% or less, or 12.1% or less, based on the total weight of the particle. More preferably, the atomic oxygen content is 10% or more, and 35% or less, such as 25% or less, 23% or less, 22% or less, 21% or less, 15% or less, 14% or less, and 13% or less. Even more preferably, 12 wt. % or more, and 13% or less, such as 12.9% or less, 12.7% or less, 12.2% or less, and 12.1% or less. The atomic oxygen content in the particle is controlled by the difference between the atomic mass of the metal and the atomic mass or molecular weight of (other) oxygen-containing species present. For example, when the particle comprises scandium hydroxide, yttrium hydroxide, samarium hydroxide, gadolinium hydroxide, dysprosium hydroxide, holmium hydroxide, ytterbium hydroxide, or lutetium hydroxide, the atomic oxygen content may be about 50 wt. %, 34.3 wt. %, 23.8 wt. %, 23.0 wt. %, 22.5 wt. %, 22.2 wt. %, 21.4 wt. %, or 21.2 wt. %, respectively, based on the total weight of the particle.

The lanthanide hydroxide as part of the particle according to the invention may further comprise one or more metal complexes, wherein the one or more metal complexes comprise one or more Lewis bases, such as monodentate ligands and/or chelating ligands. In particular, the one or more metal complexes comprise a metal as described herein.

According to the invention, the Lewis base preferably is an oxygenated or nitrogenous Lewis base. The Lewis base may be susceptible to hydrolysis. In particular, the Lewis base comprises hydride, hydroxide, oxide (oxygen), water, acetate, sulphate, carbonate, phosphate, alcohols, ketones, such as β-dicarbonyl species exhibiting the keto-enol tautomerism (e.g. acetylacetone), carboxylates, and/or hydroxyl acids. Preferably, hydride, hydroxide, oxide, water, acetate, sulfate, carbonate, phosphate, ketones, in particular β-dicarbonyl species exhibiting the keto-enol tautomerism (e.g. acetyl acetone), ethylene diamine, oxalate, dimethyl glyoximate, acetyl acetonate, methyl acetoacetate, and/or ethyl acetoacetate are selected. More preferably, the Lewis base is oxide, hydroxide, β-dicarbonyl species exhibiting the keto-enol tautomerism (e.g. acetyl acetone), acetyl acetonate, ethylene diamine, oxalate, dimethyl glyoximate, methyl acetoacetate, and/or ethyl acetoacetate. Even more preferably, the Lewis base is oxide and/or hydroxide.

The particle according to the invention may further comprise a binder for the formation of the particle. The binder may have the additional properties of a stabiliser. The binder may function as a polymer matrix, comprising polymeric material, such as poly(L-lactic acid).

An advantage of using the particle according to the invention is that the oxygen in the oxygen based carrier, such as the above Lewis bases, functions as a neutron moderator, which is relatively stable against neutron irradiation. Oxygen is also typically resistant to modification of its shape (i.e. keeps the shape). Further, the surface of the oxygen material may be functionalised according to known methods in the art.

The particle of the invention has an average particle diameter in the range of 5 nm to 400 μm. In particular, the average particle diameter of the particle is 5 nm or more, and 75 μm or less, such as 55 μm or less, 30 μm or less, 15 μm or less, and 10 μm or less. Preferably, the average particle diameter is 5 nm or more, and 10 μm or less, such as 1 μm or less, 0.5 μm or less, and 0.1 μm or less. The average particle diameter, as used herein, is typically the value that can be determined with a multisizer for microparticles and a Malvern ALV CGS-3, unless otherwise indicated. Typically, the diameter of the particle is calculated form the peak width of the diffraction pattern of a specific component using the Scherrer equation. The diameter of the particle may also be suitably determined with other methods, such as transmission electron microscopy (TEM), scanning electron microscopy (SEM), or optical microscopy. The diameter of the particle refers to the largest dimension of the particle. Table 1 shows common and preferred selected average particle diameter ranges for the particle when used in Enhanced Permeability and Retention (EPR) targeting, sentinel node procedure, intratumoural injection, radioembolisation, embolisation, and radiation synovectomy. Concerning intratumoural injection, the average particle diameter is more preferably 5-30 μm, and even more preferably 5-15 μm. With radioembolisation the average particle diameter is more preferably 20-40 μm.

TABLE 1

| | Common average | Preferred average |
|---|---|---|
| | particle diameter | particle diameter |
| EPR targeting | 5 nm-500 nm | 10 nm-200 nm |
| Sentinel node procedure | 50 nm-3000 nm | 50 nm-1000 nm |
| Intratumoral injection | 500 nm-80 μm | 1 μm-40 μm |
| Radioembolisation | 15 μm-60 μm | 20 μm-60 μm |
| Embolisation | 15 μm-400 μm | 80 μm-300 μm |
| Radiation synovectomy | 30 nm-50 μm | 2 μm-5 μm |

The particle as described herein may be nonradioactive or radioactive, depending on the application of the particle. In an embodiment, the particle is not radioactive. In another embodiment, the particle is (made or being) radioactive.

In the case the particle is made radioactive, the particle comprises one or more radioactive elements (i.e. radionuclides) that emit radiation suitable for diagnosis and/or therapy. The radionuclides are (rapidly) decaying (half-life of a few minutes to a few weeks) to, in general, a stable nuclide after emitting ionising radiation. The most common types of ionising radiation are (1) alpha(α)-particles, (2) β-particles, i.e. electrons that are emitted from the atomic nucleus, (3) gamma-(γ)rays and/or X-rays. For therapeutic purposes, radionuclides that emit β- or electron radiation, and in some exceptional applications α-radiation, are applied. The radiation will damage DNA in the cell which results in cell death.

Often, the radionuclide is attached to a carrier material that has a specific function or size which brings the radionuclide to a specific organ or tissue. The design of these carrier compounds is based solely upon physiological function of the target tissue or organ. This carrier material is often an endogenous compound, which is naturally present in the human, humanoid or nonhuman body. The carrier compounds of the invention are the Lewis bases as described herein in the case that the binder is absent. The particle of the invention will be adapted in diameter and composition for its specific application. Preferably, the particle of the invention is stable when brought into contact with carrier material as described herein.

In particular, the particle of the invention may be biodegradable. A biodegradable particle allows degradation in a human, humanoid or nonhuman body after it has been used, for instance for radiotherapy and/or magnetic resonance imaging.

The invention provides the particle(s) according to the invention for use in medical applications. In an embodiment, the particle of the invention is provided for use as a medicament or as a medical device.

The term “medical applications” as used herein is meant to include methods for treatment of the human or animal body, such as radiation synovectomy (e.g. rheumatoid arthritis), intratumoural injection, bone fractures to decrease inflammation, embolization (e.g. radioembolisation), sentinel node procedure, EPR targeting, and brain treatment procedures (e.g. epilepsy). Humans, humanoids, and/or non-humans, such as domesticised animals (i.e. pets, livestock, zoo animals, equines, etc.), may be subjected to the medical applications.

In an embodiment, the particle of the invention is used in a method of surgery, therapy and/or in vivo diagnostics. The method of surgery, therapy and/or in vivo diagnostics is a method of detecting and/or treating one or more cancers, particularly in the treatment of one or more cancers selected from the group consisting brain, pancreas, lymph, lung, head, neck, prostate, breast, liver, intestines, thyroid, stomach and kidney cancers, and more in particular metastases, by administering the particle. The particle may suitably be administered to cancers of the brain, pancreas, intestines, thyroid, stomach, head and/or neck, lung and/or breast cancers and/or tumours via an (intratumoural) injection. The particle may also be suitably administered to cancers of the liver, kidney, pancreas, brain, lung and/or breast via a catheter (for example radioembolisation of liver tumours). The particle may also be suitably administered by (direct or intravenous) injection, infusion, a patch on the skin of an individual (i.e. a skin patch), etc.

In an embodiment, the form of radiotherapy used is radioembolisation. Radioembolisation is a treatment which combines radiotherapy with embolisation. Typically, the treatment comprises administering (i.e. delivering) the particle used according to the invention, for instance via catheterisation, into the arterial blood supply of an organ to be treated (i.e. intra-arterial injection), whereby said particle becomes entrapped in the small blood vessels of the target organ and irradiates the organ. In an alternate form of administration the particle may be injected directly into a target organ or a solid tumour to be treated (i.e. intratumoural injection). The person skilled in the art, however, will appreciate that the administration of the particle used according to the method of the invention may be by any suitable means and preferably by delivery to the relevant artery. The particle may be administered by single or multiple doses, until the desired level of radiation is reached. Preferably, the particle is administered as a suspension, as described herein below.

The particle according to the invention in the method of detecting and/or treating a cancer, typically tends to accumulate in cancer tissue substantially more than it does in normal tissues due to the enhanced permeability and retention (EPR) effect, particularly when the particle has a size of 5 nm to 2 μm and more in particular 5 nm to 0.9 μm. This phenomenon may be a consequence of the rapid growth of cancer cells, which stimulates the production of blood vessels.

The invention further provides the particle as described herein for use in the treatment of cancer, in particular one or more cancers selected from the group consisting of cancer of the brain, pancreas, lymph, lung, head, neck, prostate, breast, liver, intestines, thyroid, stomach, and kidney. The particle as described herein may be used in the preparation of a medicament for treating cancer, in particular one or more cancers selected from the group consisting of cancer of the brain, pancreas, lymph, lung, head, neck, prostate, breast, liver, intestines, thyroid, stomach, or kidney. Preferably, the cancer is cancer of the pancreas or liver.

In another embodiment, the invention provides a method of treating one or more cancers in a subject, comprising administering to the subject the particle according to the invention. The administering of the particle according to the invention to the subject may be performed for a time sufficient to treat the one or more cancers. In particular, the one or more treated cancers may be selected from the group consisting of cancer of the brain, pancreas, lymph, lung, head, neck, prostate, breast, liver, intestines, thyroid, stomach, and kidney. Preferably, the subject is in need of the method of treating one or more cancers as described herein and/or the one or more cancers is cancer of the pancreas and/or liver.

In a further embodiment, the invention provides the particle according to the invention for use in the diagnosis of a disease. The particle as described herein may be used in the preparation of a medicament for diagnosing a disease. In particular, the disease may be cancer, such as cancer of the brain, pancreas, lymph, lung, head and neck, prostate, breast, liver, intestines, thyroid, stomach, and/or kidney. Preferably, the cancer is cancer of the pancreas, brain, head-and-neck, and/or liver.

In another embodiment, the invention provides a method of diagnosing a disease in a subject, comprising administering to the subject the particle according to the invention. The administering of the particle according to the invention to the subject may be performed for a time sufficient to diagnose the disease. In particular, the disease may be cancer, such as cancer of the brain, pancreas, lymph, lung, head, neck, prostate, breast, liver, intestines, thyroid, stomach, and/or kidney. Preferably, the subject is in need of the method of disease, such as cancer, as described herein and/or the cancer is cancer of the pancreas and/or liver.

In another embodiment, the particle of the invention is used as a medicament, such as a pharmaceutical. In particular, the particle is used in the preparation of a pharmaceutical, preferably for the treatment of a medical disorder (i.e. a disease or condition, such as cancer). The particle according to the invention may be used for treating a medical disorder, in particular cancer. The cancer may be located in the brain, pancreas, lymph, lung, head, neck, prostate, breast, liver, intestines, thyroid, stomach, and/or kidney. Preferably, the cancer is cancer of the pancreas, brain, head-and-neck, and/or liver.

In another embodiment, the particle of the invention is used, preferably as a medicament, in (a method for) the treatment of the human, humanoid and/or nonhuman body.

In yet another embodiment, the particle of the invention is used in a method of treatment, the treatment being a method of surgery, therapy and/or in vivo diagnostics. More in particular, the method of surgery, therapy and/or in vivo diagnostics comprises:
i) imaging, such as magnetic resonance imaging, nuclear scanning imaging, X-ray imaging, positron emission tomography imaging, single-photon emission computed tomography imaging, X-ray computed tomography imaging, dual energy computed tomography, Cherenkov luminescence imaging, scintigraphy imaging, ultrasound, and/or fluorescent imaging;
ii) drug delivery;
iii) cellular labeling, and/or
iv) radiotherapy.

The particle of the invention is capable of at least in part disturbing a magnetic field. The particle can be detected by a nonradioactive scanning method, such as medical imaging, such as Computed Tomography (CT), dual energy CT, Cherenkov Luminescence Imaging (CLI), Magnetic Resonance Imaging (MRI), Positron Emission Tomography (PET), Single-Photon Emission Computerised Tomography (SPECT), and the like.

Nuclear imaging, or nuclear scanning imaging, is extremely sensitive to abnormalities in organ structure or function. The radioactive diagnostic compounds can identify abnormalities early in the progression of a disease, long before clinical problems become manifest. Moreover, radiopharmaceuticals comprise the unique ability that they can provide a treatment option by exchanging the diagnostic nuclide for a therapeutic one but using the same carrier. With most of the compounds only the radioactivity of the radiopharmaceutical (e.g. lanthanide) has to be increased as these radionuclides emit often both β- and γ-radiation for therapy and diagnosis, respectively. The distribution and biological half-life of the specific therapeutic compound are then mostly very similar to that of the diagnostic compound. For example, the use of ¹⁶⁶Ho particles according to the invention for diagnostic application in a screening dose (or scout dose) will contain typically 1-30 MBq/mg, such as 2-10 MBq/mg and 3-7 MBq/mg. The particle can also be nonradioactive in diagnostic applications using CT and/or MR imaging.

For treatment of different types of tumours, e.g. radioembolisation of hepatocellular carcinoma (HCC), liver metastases, bone metastases, a treatment dose may typically contain 2-60 MBq/mg, such as 5-30 MBq/mg and 6-12 MBq/mg. For intratumoural and radiosegmentectomy of tumours, a treatment dose may typically contain 1-200 MBq/mg, such as 3-100 MBq/mg, 5-60 MBq/mg, or 6-15 MBq/mg.

In general, the amount of activity/mg for a screening dose, and a treatment dose, for example for diagnostic applications, and for therapeutic treatments, such as radioembolization and intratumoral injection, respectively, may vary depending on the dose and number of the particles.

The particle of the invention may be present in a suspension. The invention provides a suspension comprising the particle according to the invention, the suspension being a therapeutic suspension, e.g., an active therapeutic suspension, diagnostic suspension or a scanning suspension, such as a magnetic resonance imaging scanning suspension or a nuclear scanning suspension.

The term “suspension” as used herein, is meant to include dispersions. Typically, the suspension comprises the particle and a (carrier) fluid or gel. The suspension may comprise one or more buffering agents, such as phosphate buffered saline (PBS) and succinic acid, toxicity adjusting agents, such as sodium chloride and dextrose, solubilising agents, such as pluronic and polysorbates 20 or 80 (i.e. TWEEN 20 and 80), complexing and dispersing agents, such as cyclodextrins, flocculating/suspending agents, such as carboxymethylcellulose, gelatin, hyaluronic acid, wetting agents, such as surfactants like glycerin, PEG and pluronics, preservatives, such as parabens and thiomersal (or thimerosal), antioxidants, such as ascorbic acid and tocopherol, chelating agents, such as ethylene diamine tetraacetic acid (EDTA), and/or contrast agents, such as iomeprol (Iomeron®), iodixanol (Visipaque®) or iopamidol (Isovue®), or MRI contrast agents such as gadobutrol (Gadovist®) and gadoterate meglumine (Dotarem®). Suitably, the suspension comprises one or more (carrier) fluids, wherein the one or more (carrier) fluids comprise aqueous solutions, such as a saline solution (i.e. sodium chloride in water), a PBS solution, a tris-buffered saline (TBS) solution, or blood (e.g. of human or animal origin). Suitable examples of gel for use in the suspension are a dextran, gelatin (starch) and/or hyaluronic acid.

The suspension of the invention suitably comprises a scanning suspension, whereby the particle(s) is (are) capable of at least in part disturbing a magnetic field. The particle(s) can be detected by radioactive or nonradioactive scanning methods (tomography), such as magnetic resonance imaging (MRI), positron emission tomography (PET), single-photon emission computed tomography (SPECT), computed tomography (CT), e.g., dual energy CT and dual-enhanced Cardiovascular Computed Tomography (CCT), Cherenkov luminescence imaging (CLI), and the like. Preferably the scanning suspension comprises an MRI, CLI, CT, dual energy CT, or SPECT, scanning suspension, or a nuclear scanning suspension.

The suspension suitably comprises particle(s) of which the composition is capable of essentially maintaining its/their structure during neutron activation (i.e. neutron irradiation).

In an embodiment, the use of the particle of the invention for the preparation of a scanning suspension is provided. Preferably, the scanning image obtained by using the particle as described herein is an MRI, CLI, CT, dual energy CT, or SPECT, scanning image, or a nuclear scanning image.

The scanning suspension of the invention is suitable for determining a flowing behaviour of the particle according to the invention.

The scanning suspension is also suitable for detecting a malignancy, e.g. a tumour. In particular, the tumour comprises a liver metastasis or pancreas metastasis.

In an embodiment of the invention, a method is provided for detecting a malignancy, e.g. a tumour, comprising:
i) administering to an individual a scanning suspension comprising a particle in accordance with the invention which is capable of at least in part disturbing a magnetic field;
ii) obtaining a scanning image, and
iii) determining whether the image reveals the presence of a tumor.

The scanning image may be obtained with medical imaging. Preferably, the scanning image is a tomographic image that is generated with CLI, CT, dual energy CT, MRI, PET, SPECT, or the like. More preferably, the image is generated with dual energy CT.

The suspension according to the invention can be used as such as a therapeutic composition and/or diagnostic composition. In addition, the suspension can be used for the preparation of a diagnostic composition. The suspension can be nonradioactive or radioactive.

The invention also relates to a composition comprising the particle according to the invention, or the suspension of the invention, wherein the particle of the particle present in the suspension further comprises a pharmaceutically acceptable carrier, diluent and/or excipient. The composition as described herein may be a pharmaceutical composition.

In an embodiment, the composition of the invention is a therapeutic composition which comprises a radioactive particle according to the invention. Such a therapeutic composition can suitably be brought in the form of a suspension before it is administered to an individual. Such therapeutic composition has the advantage that it requires a shorter neutron activation time and that it displays a higher specific activity. In addition, a reduced amount of particles need to be administered to the individual, or patient.

The particle of the invention can be directly generated using a radioactive component, such as radioactive holmium. Preferably, a nonradioactive particle of the invention is firstly generated, followed by irradiation of the particle which decreases unnecessary exposure to radiation of personnel. This can avoid the use of high doses of radioactive components and the need for specially equipped (expensive) facilities, such as hot cells and transport facilities. In particular, the radioactive component may be a therapeutically active compound.

In an embodiment, the above therapeutic composition comprises a particle of the invention, which particle is provided with at least one therapeutically active compound, for instance capable of treating a tumour. Such a therapeutic composition is for instance capable of treating a tumour simultaneously by radiotherapy and with a therapeutic action of the therapeutically active compound.

In another embodiment, a nonradioactive therapeutic composition is provided, comprising a nonradioactive particle of the invention which is provided with at least one therapeutically active compound, for instance, capable of treating a tumour.

In another embodiment, the use of the particle according to the invention for detecting a malignancy, such as a tumour, is provided. Such a tumour can be detected without the need of using radioactive material. Alternatively, the particle with low radioactivity can be used. After a tumour has been detected, the tumour can be treated with a therapeutic composition as described herein comprising the same kind of particles as the scanning suspension. In such a therapeutic composition, however, the particles are preferably rendered radioactive. Despite the difference in radioactivity, the particles of the diagnostic composition for detecting the tumour and particles of the therapeutic composition can be chemically the same.

In an embodiment, a kit-of-parts is provided wherein the diagnostic composition comprises the suspension according to the invention.

In another embodiment, a kit-of-parts is provided comprising a diagnostic composition and therapeutic composition, the diagnostic composition and the therapeutic composition comprising particles with essentially the same chemical structure which are capable of at least in part disturbing a magnetic field, wherein the particles comprise a diameter of at least 5 nm, wherein the therapeutic composition comprises a particle of the invention which is provided with at least one therapeutically active compound. The distribution of the therapeutic composition can be followed over time using a scanning method, such as tomographic scanning methods, e.g., CLI, CT, dual energy CT, MRI, PET, SPECT, and the like. In yet a further embodiment, the therapeutic composition is essentially nonradioactive.

The particle of the invention relates to a method of preparing the particle according to the invention, comprising:
i) adding at least one metal particle to a salt solution to form a mixture;
ii) stirring the mixture to form the particle, and
iii) recovering from at least part of the mixture of ii) the particle.

In particular, the method of preparing the particle of the invention, as described above, provides the particle as described herein.

The metal particle can be prepared by using different types of processes. Suitable preparation processes include microfluidics, membrane emulsification, solvent evaporation processes, solvent extraction processes, spray-drying processes, and inkjet printing processes. Preferably, the metal particle is made by solvent evaporation. The metal particle may comprise one or more metals and one or more Lewis bases, as described herein, such as holmium and acetyl acetonate. With the method, the metal particle undergoes a physical and/or chemical modification, in particular a chemical modification, resulting in the particle according to the invention. The modification may be the result of for example ionic exchange and/or hydrolysis.

The method of preparing the particle according to the invention, as described herein, may further comprise a washing step to be carried out after iii). The washing step comprises washing the recovered particle with a solvent as described below by, for example, centrifugation. Preferably, the recovered particle is washed with water.

The method of preparing the particle according to the invention, as described herein, may further comprise a drying step. In particular, the drying step is performed after iii). In the case the method of preparing the particle comprises a washing step, such as the washing step described above, the method may further comprise a drying step to be carried out after the washing step. The drying step comprises drying the (washed) particle, such as by drying in a (vacuum) oven or by freeze drying. The drying step may be performed at a temperature from −80° C. up to 100° C., such as between 10-80° C., and 15-50° C. Preferably, the drying step is performed at room temperature.

The method of preparing the particle according to the invention, as described herein, may further comprise a heat treating step. The particle may be (further) modified through the heat treatment. The heat treatment may be performed at a heating rate, such as 0.1-20° C. per minute, from about room temperature to 1000° C. When the particle is subjected to the heat treatment, the particle may be (chemically) modified. For example, when particles comprising lanthanide hydroxide are subjected to the heat treatment, particles comprising lanthanide oxide may form.

In an embodiment, a method is provided for preparing the particle of the invention, comprising:
i) adding at least one metal particle to a salt solution to form a mixture;
ii) stirring the mixture to form the particle;
iii) recovering from at least part of the mixture of ii) the particle;
iv) heat treating at least part of the particle of iii).

The method may provide the formation of the particle of the invention and/or a (chemically) modified particle of the invention, such as a particle comprising lanthanide oxide, during and/or after the heat treatment step iv).

The invention also relates to a particle prepared by the method as described herein, wherein the method further comprises a heat treatment step, wherein the particle comprises a metal oxide. The average particle diameter of the particle is preferably in the range of 5 nm-400 μm. In particular, the heat treatment step is the heat treating step as described herein. The particle may be a nanoparticle or a microparticle. The particle preferably is a microparticle. The particle comprising a metal oxide, such as a lanthanide oxide, as described herein, may have a shape as described herein. In particular, the particle is spherical.

In particular, the method of preparing the particle of the invention, as described above, provides the particle of the invention. The method may further comprise the above-mentioned drying step and/or washing step and/or the heating treatment.

The metal particle may comprise a metal complex as described above, for example a metal hydroxide, such as lanthanide hydroxide. In a particular embodiment, the metal particle comprises metal acetyl acetonate, for example lanthanide acetyl acetonate, such as holmium acetyl acetonate.

The salt solution may comprise any ionic compound at least in part dissolved in at least one solvent. In particular, the salt solution comprises a hydroxide salt, such as lithium hydroxide, sodium hydroxide, or potassium hydroxide. The solvent may be polar and protic, and may comprise one or more selected from the group consisting of ammonia, t-butanol, n-butanol, n-propanol, iso-propanol, nitromethane, ethanol, methanol, 2-methoxyethanol, acetic acid, formic acid, and water.

In an embodiment, the salt solution of the method of preparing the particle according to the invention comprises a hydroxide salt, such as sodium hydroxide, at least in part dissolved in a solvent, the solvent comprising water. The acidity (or pH) is a parameter of the salt solution. The salt solution may have a pH value of 7 or higher, such as 8 or higher, 9 or higher, or 10 or higher. Preferably, the salt solution has a pH value of at least 12, such as 13.5. In case the pH of the solution is below 8, the reaction might not occur. When the pH is at least 12, the reaction time will significantly decrease.

In an embodiment, the method of preparing the particle of the invention, as described herein comprises the addition of metal acetyl acetonate particle to a hydroxide salt solution, such as sodium hydroxide in water. Preferably, the pH of the salt solution is 12 or higher, such as 13.5, because hydrolysis of acetyl acetone is highly favourable at such pH. Under these conditions, metal acetyl acetone is at least partially converted to metal hydroxide.

The invention further provides a method of obtaining a scanning image, comprising:
i) administering to a human, humanoid, or nonhuman the suspension according to the invention, and subsequently
ii) generating a scanning image of the human, humanoid, or nonhuman.

In particular, the scanning image is a tomographic image. Preferably, the tomographic image is generated with CLI, CT, dual energy CT, MRI, PET and/or SPECT. More preferably, the image is generated with dual energy CT.

Magnetic resonance imaging provides information of the internal status of an individual. A contrast agent is often used in order to be capable of obtaining a scanning image. For instance iron and gadolinium, preferably in the form of ferrite particles and gadolinium-diethylamintriamine pentaacetic acid (DTPA) complexes, are often used in contrast agents for magnetic resonance imaging scanning. This way, a good impression can be obtained of internal disorders, like the presence of (a) tumour(s).

After diagnosis, a treatment is often started involving administration of a composition, e.g. a pharmaceutical or therapeutic composition, to a subject (individual, patient). If is often important to monitor the status of a patient during treatment as well. For instance the course of a treatment and targeting of a drug can be monitored, as well as possible side effects which may imply a need for terminating, or temporarily interrupting, a certain treatment.

Sometimes local treatment in only a specific part of the body is preferred. For instance, tumour growth can sometimes be counteracted by internal radiotherapy comprising administration of radioactive particles to an individual. If the radioactive particles accumulate inside and/or around the tumour, specific local treatment is possible.

In an embodiment a method is provided for treating a subject, comprising:
i) administering to the subject a diagnostic composition or scanning composition, comprising the particle as described herein, the suspension as described herein, or the composition as described herein, wherein the particle is capable of at least in part disturbing a magnetic field;
ii) obtaining a scanning image of the subject;
iii) determining the distribution of the particle within the subject;
iv) administering to the subject a therapeutic composition comprising the particle as described herein, the suspension as described herein, or the composition as described herein, wherein the particle in the therapeutic composition is preferably more radioactive than the particle in the diagnostic composition or scanning composition.

The scanning image of a subject may be obtained with medical imaging, e.g., tomographic imaging techniques, such as CLI, CT, dual energy CT, MRI, PET, SPECT, and the like.

The invention provides the particle as described herein for use in the treatment of a subject, the treatment comprising:
i) administering to the subject a diagnostic composition or scanning composition, comprising the particle as described herein, the suspension as described herein, or the composition as described herein, wherein the particle is capable of at least in part disturbing a magnetic field;
ii) obtaining a scanning image of the subject;
iii) determining the distribution of the particle within the subject;
iv) administering to the subject a therapeutic composition comprising the particle as described herein, the suspension as described herein, or the composition as described herein, wherein the particle in the therapeutic composition is preferably more radioactive than the particle in the diagnostic composition or scanning composition.

The scanning image of a subject may be obtained with medical imaging, e.g., tomographic imaging techniques, such as CLI, CT, dual energy CT, MRI, PET, SPECT, and the like.

In an embodiment, the particle in the therapeutic composition is radioactive while the particle in the diagnostic composition or scanning composition is not radioactive.

The diagnostic composition or scanning composition may comprise an amount of the particle as described herein which is higher than the amount of the particle present in the therapeutic composition, or vice versa. In case the particle is prepared by the method as described herein, e.g. the particle comprising a metal oxide complex, both the diagnostic composition of scanning composition and the therapeutic composition require a lower amount of particles, when compared to the case the particle comprises a metal hydroxide complex.

In an embodiment, the particles as described herein for use in the treatment of a subject, the treatment comprising diagnosing and/or screening. The particles, or screening dose, may be either radioactive or nonradioactive. A radioactive screening dose, or radioactive particles, can for example be used to determine lung shunt, lung dose, blood backflow, uptake in (other) organs, etc. Whereas nonradioactive particles can be used for imaging with CT, dual energy CT, CLI, PET, SPECT, and MRI. Herewith, the particles for imaging can be used, for example to predict the (eventual) distribution of the (radioactive) particles for a treatment on a subject, comprising a therapeutic, cosmetic and/or surgical treatment. In other words, when the particles for imaging have the same or similar properties to the particles for treatment, the distribution of the particles can be predicted.

The invention provides the particle as described herein capable of at least in part disturbing a magnetic field for use in the treatment of a tumour in a subject, wherein the dosage of the particle is derived from a scanning image obtained with a scanning suspension, such as the suspension as described herein, comprising particles capable of at least in part disturbing a magnetic field with the same chemical structure as the particle, based on the distribution of the particles of the scanning suspension with the same chemical structure within the subject, and wherein the particle for use in the treatment of the tumour preferably exhibits a higher amount of radioactivity per particle than the particles used for obtaining the scanning image. The tumour may comprise, for example any type of tumour and/or cancer as described herein. Since the particle as described herein is used for obtaining a scanning image as well as for radiotherapy, a method or use of the invention is preferably provided wherein the particle comprises a composition capable of essentially maintaining its structure during irradiation for at least 0.5 hour, preferably for at least about 1 hour, such as up to 10 hours, with a neutron flux of e.g. 4.1·10¹⁷ m⁻²·s⁻¹. The distribution of the particle may be followed over time. The scanning image may be obtained with tomographic imaging, such as CLI, CT, dual energy CT, MRI, PET, SPECT, or the like.

The invention further provides a use of the particle as described herein in medical imaging, preferably CLI, CT, dual energy CT, MRI, PET, SPECT, and the like, more preferably dual energy CT.

The invention has been described by reference to various embodiments, and methods. The skilled person understands that features of various embodiments and methods can be combined with each other.

All references cited herein are hereby completely incorporated by reference to the same extent as if each reference were individually and specifically indicated to be incorporated by reference and were set forth in its entirety herein.

The use of the terms “a” and “an” and “the” and similar referents in the context of describing the invention (especially in the context of the claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. For the purpose of the description and of the appended claims, except where otherwise indicated, all numbers expressing amounts, quantities, percentages, and so forth, are to be understood as being modified in all instances by the term “about”. Also, all ranges include any combination of the maximum and minimum points disclosed and include any intermediate ranges therein, which may or may not be specifically enumerated herein.

Preferred embodiments of this invention are described herein. Variation of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject-matter recited in the claims appended hereto as permitted by applicable law. Moreover, any combination of the above-described elements in all possible variations thereof is encompassed by the invention unless otherwise indicated herein or otherwise clearly contradicted by context. The claims are to be construed to include alternative embodiments to the extent permitted by the prior art.

For the purpose of clarity and a concise description features are described herein as part of the same or separate embodiments, however, it will be appreciated that the scope of the invention may include embodiments having combinations of all or some of the features described.

Hereinafter, the invention will be illustrated in more detail, according to specific examples. However, the invention may be embodied in many different forms and should not be construed as being limited to the embodiments set forth herein. Rather, these example embodiments are provided so that this description will be thorough and complete, and will fully convey the scope of the invention to those skilled in the art.

### EXAMPLES

### Materials

All chemicals are commercially available and were used as obtained. Holmium chloride (HoCl₃.6H₂O; M_{w}=379.38 g/mol; 99.9%) was obtained from Metal Rare Earth Limited. Acetyl acetone (acac; ReagentPlus®; M_{w}=100.12 g/mol; >99%), polyvinyl alcohol (PVA; M_{w}=30 000-70 000 g/mol; 87-90% hydrolysed) were obtained from Sigma-Aldrich. Sodium hydroxide (pellets EMPLURA®, M_{w}=40.00 g/mol), ammonium hydroxide (EMSURE®; M_{w}=35.05 g/mol; 28-30%), chloroform (EMPROVE®, M_{w}=119.4 g/mol), were supplied by Millipore.

### Example 1

### Preparation of Holmium Hydroxide Microspheres

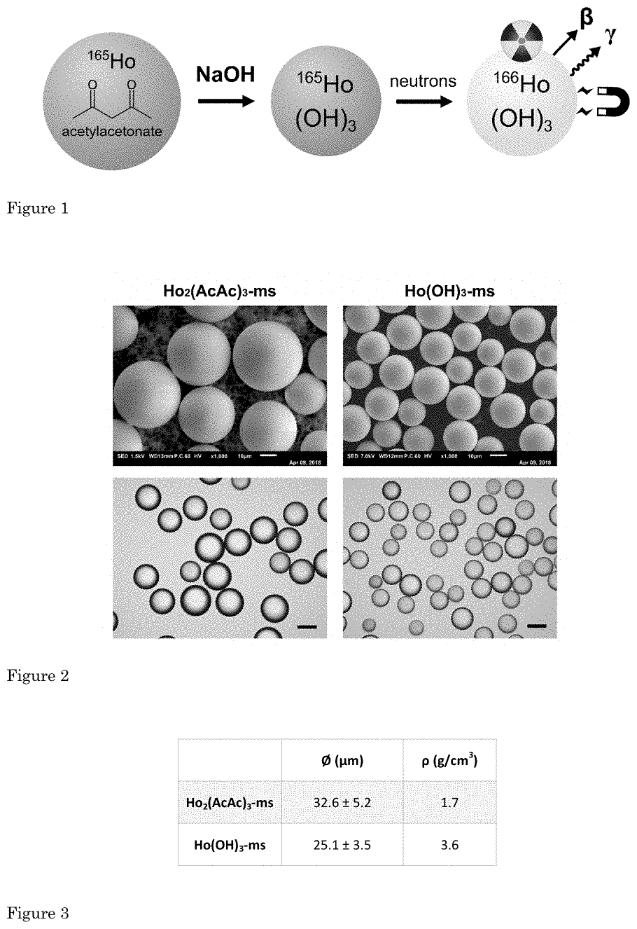

The starting material to prepare holmium hydroxide microspheres was holmium acetyl acetonate microspheres (FIGS. 1 and 2). The preparation of holmium acetyl acetonate was reported by Arranja, et al., Int. J. Pharm. 2018, 548, 73-81. A solution of crystals of holmium acetyl acetonate (10 g) dissolved in chloroform (186 g) was added to an aqueous solution of polyvinyl alcohol (1 kg water with 2% w/w polyvinyl alcohol). Overhead four blades propeller stirrers (Hei-TORQUE Value 100, Heidolph, Germany) were used to vigorously stir the mixture at 300 rpm in two litres baffled beakers to obtain an oil-in-water (o/w) emulsion. After 48 hours, the microspheres were sieved according to the desired size (20-50 μm) using an electronic sieve vibrator (TOPAS EMS 755). The sieved microspheres were dried at room temperature for 5 hours under ambient pressure, followed by vacuum drying at room temperature for 72 hours. Then, dried holmium acetyl acetonate microspheres (7 g) were added to an aqueous solution of 0.5 M sodium hydroxide (NaOH, 875 g H₂O, pH 13.5) to form holmium hydroxide microspheres. The dispersion was prepared in two litres baffled beakers and continuously stirred at 500 rpm and room temperature for 2 hours using overhead four blades propeller stirrers (Hei-TORQUE Value 100, Heidolph, Germany). After stirring, the holmium hydroxide microspheres were formed and collected into four 50 ml tubes. The microspheres were washed four times with water by centrifugation. After washing, the microspheres were dried in a vacuum oven at room temperature for 24 hours.

### Characterisation

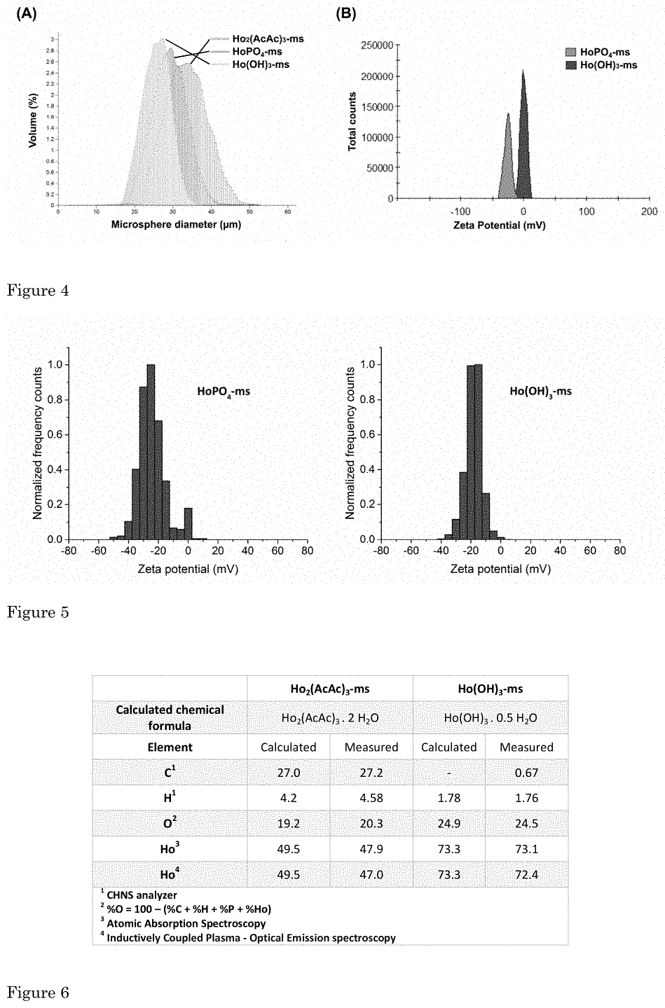

The size distributions of the starting material (holmium acetyl acetonate microspheres) and the final microspheres (holmium hydroxide microspheres; Table 1 and FIG. 4A) were determined using a Coulter counter equipped with an orifice of 100 μm (Multisizer 3, Beckman Coulter, Mijdrecht, The Netherlands). FIG. 4A further shows the determined size distribution of holmium phosphate microspheres.

An optical microscope (AE2000 Motic) was used to investigate the morphological properties of the microspheres suspended in water (sphericity and surface damages). The surface composition and smoothness of the microspheres was analysed using a Scanning Electron Microscope-Energy Dispersive X-ray Spectroscopy (SEM-EDS) (JEOL JSM-IT100, InTouchScope™, Tokyo, Japan; FIG. 2).

The zeta (ζ-)potential was determined using a Zetasizer Nano-Z Malvern Instruments) which was calibrated using a zeta potential transfer standard (DST1235, −42±4.2 mV, Malvern Instruments, UK). The samples were prepared by dispersing 25 mg of holmium phosphate microspheres or holmium hydroxide microspheres in 10 mM sodium chloride. FIG. 4B shows the comparative apparent ζ-potentials of holmium hydroxide microspheres and holmium phosphate microspheres. The pH values of the dispersions were measured (FiveEasy Plus, Mettler Toledo LE410) and were 7.0±0.2 (n=3 for each microsphere). Then, the samples were transferred into a dip cell (Universal Dip Cell Kit, ZEN 1002, Malvern Instruments, UK) and the temperature in the cell was stabilized at 25° C. for 90 seconds after which the electrophoretic mobility was determined. The ζ-potential was calculated using the Helmholtz-Smoluchowski equation (FIG. 4B). The mean zeta potential of the holmium phosphate was −27.1±2.3 mV and of the holmium hydroxide was −0.6±2.0 mV in 10 mM NaCl.

The zeta potential of the holmium phosphate and holmium hydroxide microspheres was also determined using a ZetaCompact (CAD instruments, France). The samples were prepared by dispersing approximately 50 mg of microspheres in 10 ml of water for injection (BBraun, Germany). The pHs of the dispersions were measured (FiveEasy Plus, Mettler Toledo LE410) and were 7.3±0.2 for the holmium phosphate and 7.0±0.1 for the holmium hydroxide (n=3 for each microsphere type). The samples were transferred into a quartz capillary cell and the electrophoretic mobility of individual microspheres was recorded by video microscopy. The zeta potential was then obtained using the Smoluchowski formula. The zeta potential of 500-1000 microspheres of holmium phosphate and of holmium hydroxide was obtained (FIG. 5). The mean zeta potential of the holmium phosphate was −23.8±8.9 mV and of the holmium hydroxide was −17.9±5.2 mV in water.

The density of the holmium hydroxide microspheres was determined in water using a 25 cm³ specific gravity bottle (Blaubrand NS10/19, DIN ISO 3507, Wertheim, Germany; FIG. 3) and using a sample amount of approximately 250 mg (FIG. 3).

The holmium content was determined by Inductively Coupled Plasma-Optical Emission spectroscopy (ICP-OES; FIG. 6). Before preparation of the sample for ICP-OES analysis, the microspheres were dried overnight in a vacuum oven at room temperature. Then, samples of 20 to 50 mg were dissolved in 50 ml of 2% nitric acid and the holmium concentration of the solutions was measured at three different wavelengths (339.9, 345.6 and 347.4 nm) using an Optima 4300 CV (PerkinElmer, Norwalk, USA).

The holmium content was also determined by Atomic Absorption Spectroscopy (Perkin Elmer Model AAnalyst 200) and the carbon and hydrogen contents determined with a CHNS analyzer (Elementar Model Vario Micro Cube). These elemental determinations (FIG. 6) of the holmium, carbon and hydrogen contents were performed in duplicate by Mikroanalytisches Laboratorium KOLBE (Oberhausen, Germany) and the samples were dried overnight in a vacuum oven at 100° C. The oxygen content cannot be determined accurately due to interference from the high amount of holmium, and was assumed to be the remaining component of the microspheres as no other element is expected to be present in the microspheres [% oxygen=100−(% carbon+% hydrogen+% holmium)].

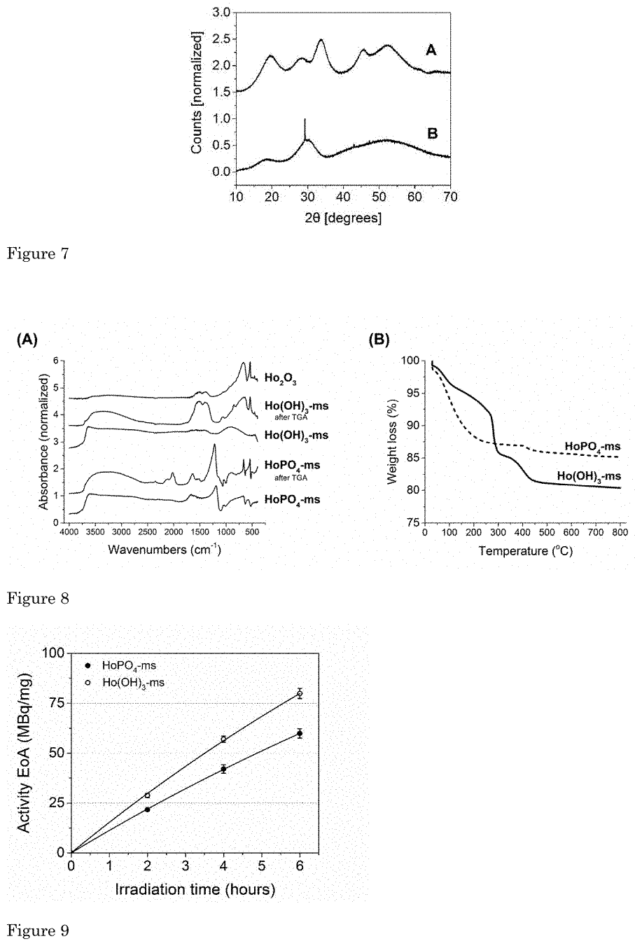

X-ray powder diffraction (XRD) patterns of the holmium hydroxide microspheres were obtained by depositing a small amount (about 5 mg) of each sample on a Si-510 wafer and analysed using a Bruker D8 Advance diffractometer in Bragg-Brentano geometry with a Lynxeye position sensitive detector (FIG. 7B). FIG. 7 further shows a comparison with the X-ray powder diffraction pattern of holmium phosphate microspheres (A).

Fourier Transform Infrared (FTIR) spectrum of the holmium hydroxide microspheres was obtained using a Nicolet 8700 FTIR spectrometer (Thermo Electron Corporation) equipped with a KBr/DLa/TGS D301 detector cooled with liquid nitrogen (FIG. 8A). FIG. 8A further shows as a comparison the FTIR spectra of holmium oxide and holmium phosphate microspheres. A small amount of the sample (5-10 mg) was pressed onto potassium bromide salt and the sample holder was stabilised for 5 minutes at 25° C. and kept at this temperature during the analysis. The FTIR spectra of the microspheres were collected at a resolution of 4 cm⁻¹ averaged over 128 scans.

Thermogravimetric analysis (TGA) of the microspheres was performed using a TGA2 Star System (Mettler Toledo; FIG. 8B). FIG. 8B further shows the TGA of holmium phosphate microspheres. Samples of 12-15 mg of microspheres were heated from 30° C. up to 800° C. in a nitrogen environment at a heating speed of 5° C./min and the weight loss was recorded. After the heat treatment, the resulting powders were also analysed by FTIR using the same conditions as described above and are shown in FIG. 8A.

### Neutron Activation

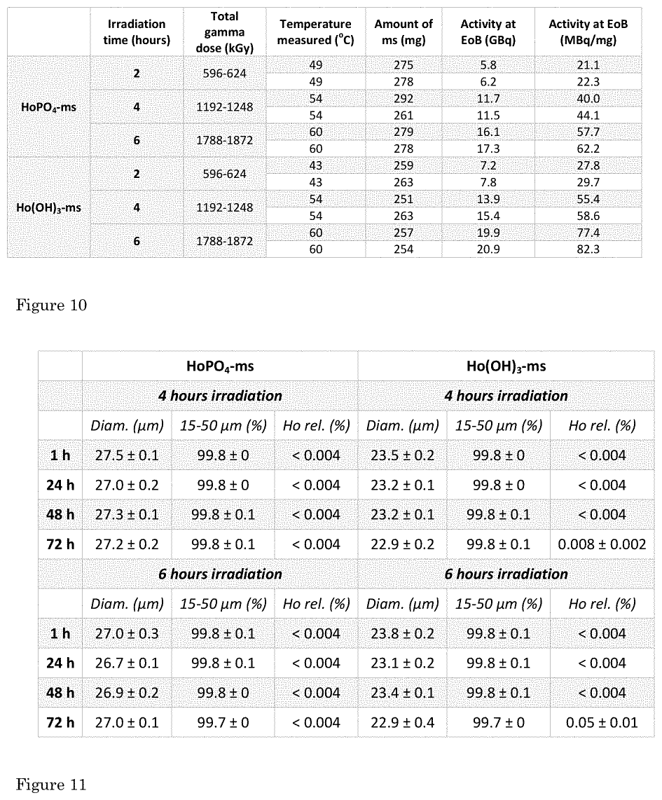

The holmium hydroxide microspheres were neutron activated in the pneumatic rabbit system (PRS) facility of the nuclear reactor research facility operational at the Department of Radiation Science and Technology of the Delft University of Technology (The Netherlands). This facility has an average neutron thermal flux of 4.72×10¹⁶ m⁻²·s⁻¹, s epithermal neutron flux of 7.87×10¹⁴ m⁻²·s⁻¹ and a fast neutrons flux of 3.27×10¹⁵ m⁻²·s⁻¹. Several amounts of microspheres (from 251 to 292 mg) were sealed in polyethylene vials which were placed into polyethylene rabbits for irradiation (Vente et al., Biomed. Microdevices 2009, 11, 763-772; Vente et al., Eur. J. Radiol. 2010, 20, 862-869). The microspheres were irradiated for 2, 4 and 6 hours (n=2) to yield radioactive holmium-166 hydroxide microspheres (¹⁶⁶Ho(OH)₃-ms); FIGS. 9 and 10). Both FIGS. 9 and 10 show, as a comparison, the data of holmium phosphate microspheres as well. During neutron bombardment, the microspheres also received a γ-dose of approximately 298 to 312 kGy per hour of irradiation. The maximum temperature reached during irradiation was monitored with temperature indicator strips (temperature points: 37° C., 40° C., 43° C., 46° C., 49° C., 54° C., 60° C., and 65° C.) that were attached to the vials immediately prior to irradiation (Digi-Sense, Cole-Parmer). The conditions of all the neutron bombardments preformed in this study are shown in FIG. 10 (this includes data from holmium phosphate microspheres).

After neutron activation, the activity of the samples at a specific time (Aₜ) was measured using a dose calibrator (VDC-404, Comecer, The Netherlands). This measurement enables the calculation of the actual activity at the end of neutron activation (i.e. end of bombardment (EoB) (A_{EoB})) by taking into account the radioactive decay after neutron activation and the measurement time, according to the following equations;

Aₜ=A_{EoB}·e^{-λt} (1)

$$( 2 ) λ = \frac{ln 2}{{T}_{1 / 2}} ,$$

λ=decay constant (s⁻¹) and T_{1/2}=half-life of the radionuclide.

The activity of the holmium hydroxide was measured when these samples decayed to 200-500 MBq/sample.

Radiochemical Purity after Neutron Activation

The holmium hydroxide microspheres that were neutron irradiated for 6 hours were analysed by gamma spectrometry after 24 and 28 days of decay time to determine the presence of radionuclide impurities, especially the longer lived radionuclides. A LG22 High Purity Germanium (HPGe) detector from Gamma Tech (Princeton, USA) and a gamma spectrum analysis software (Genie™ 2000 Ver. 3.2, Canberra, Meriden, USA) were used. Each sample was counted for 120 seconds at a defined distance from the detector. The radioactive elements that corresponded to significant energy peaks were identified.

Stability of Microspheres in Administration Fluids after Neutron Activation

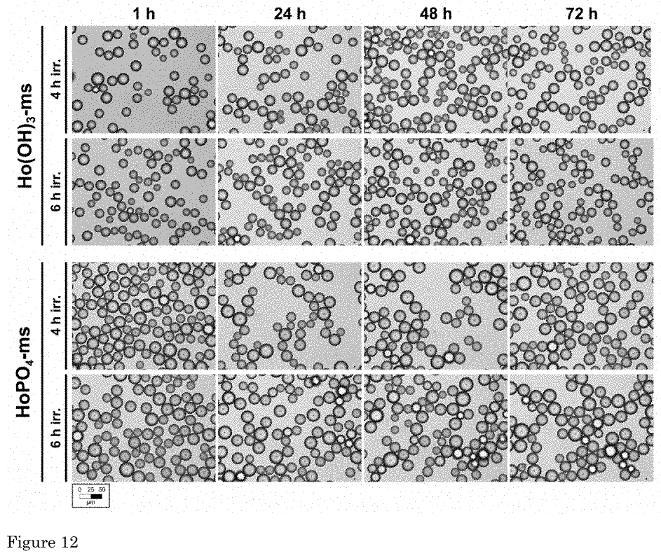

After neutron activation, the holmium hydroxide microspheres were decayed for 21 days before handling to minimise radiation exposure. Then, the holmium hydroxide microspheres were incubated with 0.9% sodium chloride (2 ml per sample) and vortexed for 10 minutes. Subsequently, the morphological properties of the microspheres were observed by optical microscopy and the size distribution was measured at predetermined time points (1, 24, 48 and 72 hours; FIG. 11). FIG. 12 shows optical microphotographs of 4 and 6 hours neutron irradiated holmium phosphate microspheres as well. Samples of the supernatant (200 μl) were collected at the same time points, diluted in 5 ml of 2% nitric acid and analysed by ICP-OES to detect possible holmium leakage (FIG. 11).

### Haemocompatibility, Haemolysis and Coagulation

One of the requirements of microspheres that will directly contact blood in certain applications, such as radiation segmentectomy or radioembolisation, is that they are haemocompatible.

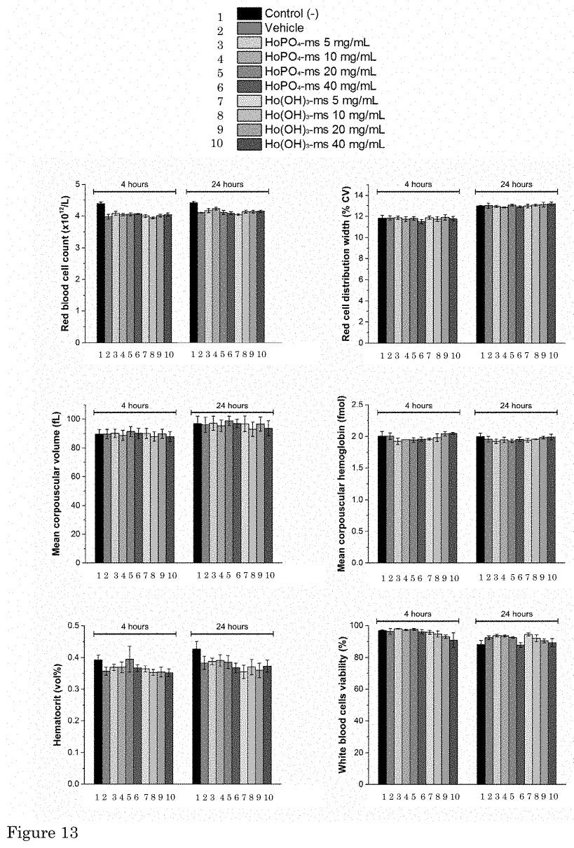

The holmium phosphate and holmium hydroxide microspheres were incubated with full human blood (concentrations ranging from 5 to 40 mg/ml), followed by analysis of the haemogram after 4 hours and 24 hours using an automated blood cell analyser (CELL-DYN Sapphire, Abbott Diagnostics, Santa Clara, Calif., USA) (FIG. 13). Statistical analysis of the haemogram results (red blood cell count, red cell distribution width, mean corpuscular volume, mean corpuscular haemoglobin concentration, haematocrit and white blood cell viability) revealed no statistically significant difference between the blood incubated with the microspheres and the respective controls (p>0.05). The holmium phosphate and holmium hydroxide microspheres did not induce alterations of the blood parameters as well as no statistically significant cytotoxicity was observed towards the white blood cells (FIG. 13).

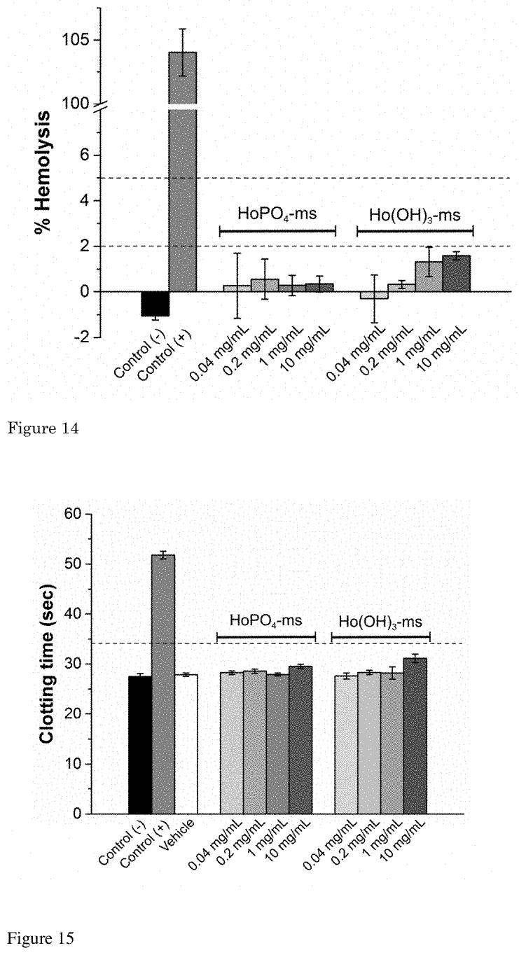

The haemolysis potential of the holmium phosphate and holmium hydroxide microspheres was determined according to the ASTM F756-00 and ASTM E2524-08. The microspheres were incubated at 37° C. with gentle mixing (VWR® mutating mixer) for 3 hours with diluted human heparinised blood at final concentrations of 0.04 mg/ml, 0.2 mg/ml, 1 mg/ml and 10 mg/ml. After incubation, the samples were centrifuged (800×g, 15 min), and the concentration of haemoglobin in a supernatant was determined. The results expressed as a percentage of haemolysis (FIG. 14) were used to evaluate the acute in vitro haemolytic properties of the microspheres. A sample with a percentage of haemolysis less than 2% is considered not haemolytic, a percentage of haemolysis between 2-5% is considered slightly haemolytic, and a result of more than 5% means the sample is haemolytic according to ASTM F756-00. FIG. 14 demonstrates that the holmium phosphate and holmium hydroxide microspheres are not haemolytic in the tested concentration range (0.04 to 10 mg/ml).

The ability of the holmium phosphate and holmium hydroxide to interact with the plasma coagulation factors of the intrinsic pathway was assessed using the activated prothrombrin time (aPTT) test. This assay evaluates the functionality of some coagulation factors (e.g., XII, XI, IX, VIII, X, V, and II). An increase of the coagulation time suggests that the material depletes or inhibits these coagulation factors. Therefore, a plasma coagulation time longer than the normal value for the aPTT test (i.e., more than 34.1 s) is considered abnormal. The holmium phosphate and holmium hydroxide microspheres were incubated with human plasma and the coagulation times after incubation with the aPTT reagent were measured. FIG. 15 shows that neither the holmium phosphate nor holmium hydroxide microspheres deplete or inhibit the coagulation factors of the intrinsic pathway in the tested concentration range (0.04 to 10 mg/ml).

### Example 2

Microspheres composed of lanthanides other than holmium, such as dysprosium and yttrium, were also prepared. The morphological properties, smoothness and surface composition of the microspheres were analysed using a Scanning Electron Microscope-Energy Dispersive X-ray Spectroscopy (SEM-EDS) (JEOL JSM-IT100, InTouchScope™, Tokyo, Japan).

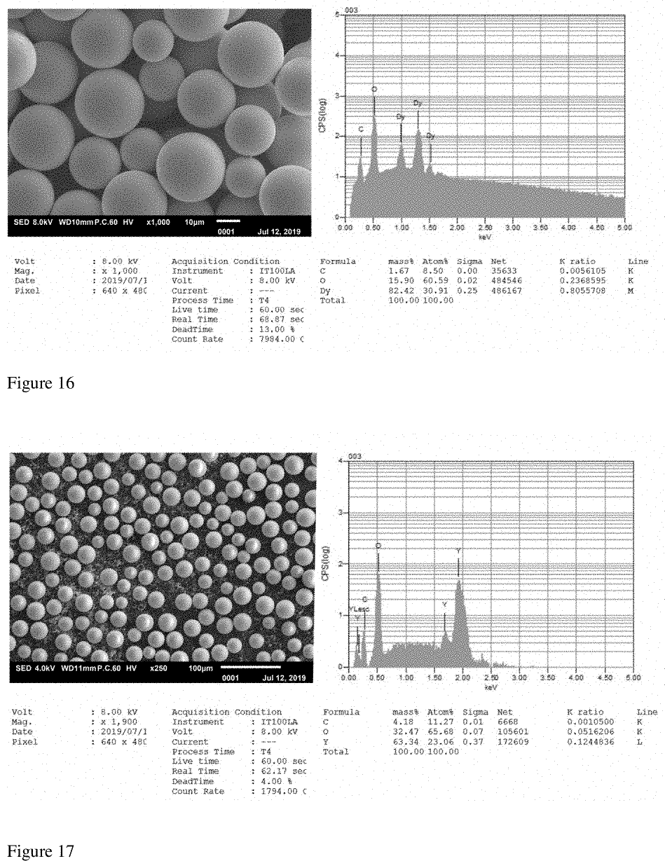

FIG. 16 depicts dysprosium hydroxide microspheres, and the respective surface elemental analysis by SEM-EDS. FIG. 17 shows a scanning electron microphotograph of the prepared yttrium hydroxide microspheres, and the corresponding surface elemental analysis by SEM-EDS.

### Example 3

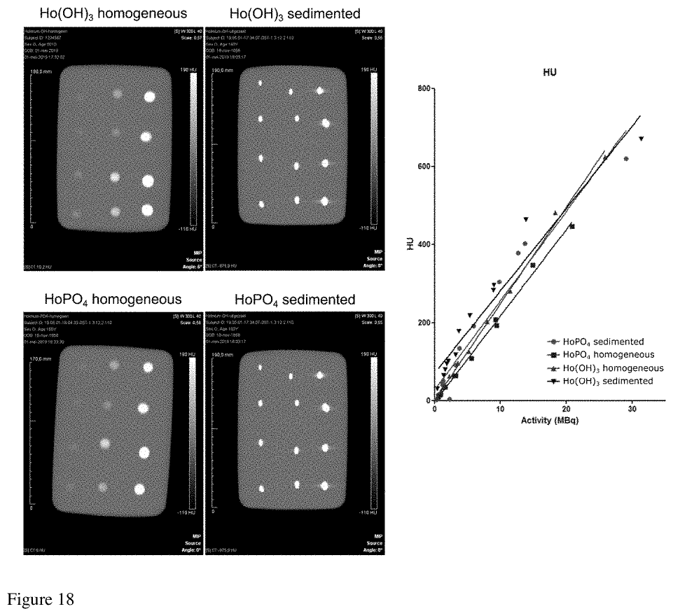

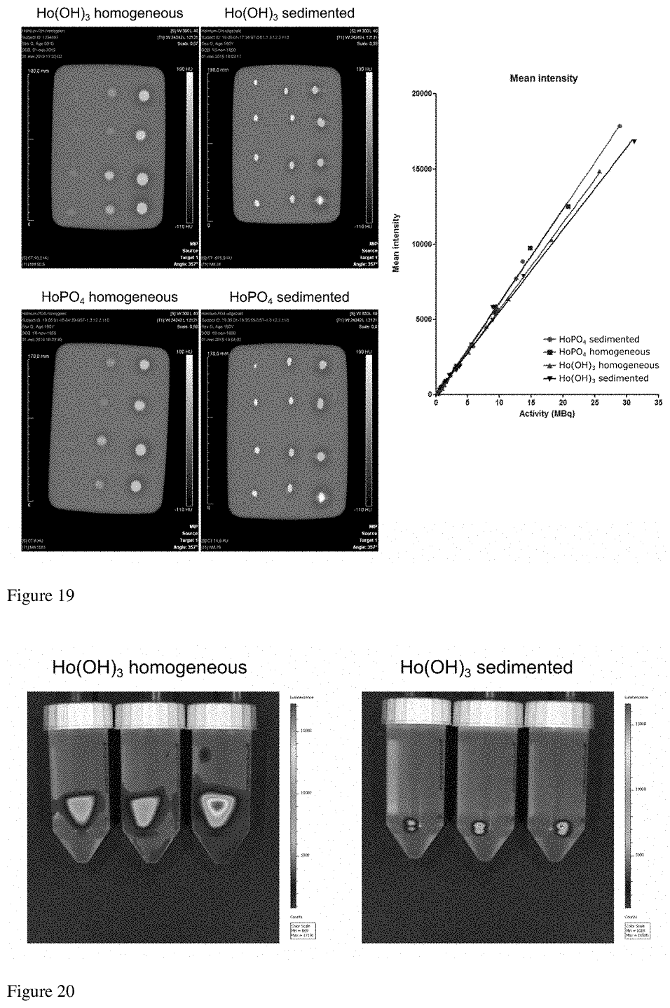

The imaging and quantification of radioactive holmium phosphate microspheres and holmium hydroxide microspheres were performed by preparing phantoms of phytagel, containing increasing concentrations of radioactive microspheres. Homogeneous distributed microspheres as well as sedimented microspheres were prepared and imaged using CT (FIG. 18), SPECT (FIG. 19) and CLI (FIG. 20). SPECT scans were acquired in a Symbia Truepoint (Siemens) and the data was processed with IRW (Inveon Research Workplace, Siemens), which resulted in good dose quantification. CLI was performed in an In Vivo Imaging System (IVIS Lumina, PerkinElmer).

1. A spherical particle comprising lanthanide hydroxide.

2. The spherical particle according to claim 1, comprising an amount of lanthanide of 15-90% by total weight of the particle.

3. The spherical particle according to claim 1, having an atomic oxygen content of 5-90%, based on a total weight of the particle.

4. The spherical particle according to claim 1, comprising one or more metals selected from the group consisting of scandium, yttrium, lanthanum, cerium, praseodymium, neodymium, promethium, samarium, europium, gadolinium, terbium, dysprosium, holmium, erbium, thulium, ytterbium, and lutetium.

5. The spherical particle according to claim 1, further comprising one or more metal complexes, wherein the one or more metal complexes comprise one or more Lewis bases.

6. The spherical particle according to claim 5, wherein the one or more Lewis bases are selected from the group consisting of monodentate ligands and chelating ligands.

7. The spherical particle according to claim 6, wherein the monodentate ligands and/or chelating ligands are selected from the group consisting of hydride, oxide, hydroxide, water, acetate, sulphate, carbonate, phosphate, ethylene diamine, oxalate, dimethyl glyoximate, methyl acetoacetate, and ethyl acetoacetate.

8. The spherical particle according to claim 1 having an average particle diameter in a range of 5 nm to 400 μm.

9. The spherical particle according to claim 1, having a sphericity of at least 0.85.

10. The spherical particle according to claim 1 being radioactive.

11. A method of preparing the spherical particle according to claim 1, comprising:
i) adding at least one metal particle to a salt solution to form a mixture;
ii) stirring the mixture to form the particle;
iii) recovering from at least part of the mixture of ii) the particle.

12. The method according to claim 11, further comprising a heat treatment step, resulting in formation of the particle comprising lanthanide oxide.

13. The spherical particle according to claim 1 which is a particle in medical applications.

14. A suspension comprising the spherical particle according to claim 1 wherein the suspension is at least one selected from the group consisting of a therapeutic suspension, a diagnostic suspension, and a scanning suspension.

15. (canceled)

16. (canceled)

17. The suspension according to claim 14, wherein the scanning suspension is a magnetic resonance imaging scanning suspension or a nuclear scanning suspension.

18. (canceled)

19. A composition comprising the particle according to claim 1, wherein the particle further comprises a pharmaceutically acceptable carrier, diluent and/or excipient.

20. A composition comprising a suspension according to claim 14, wherein the particle present in the suspension further comprises a pharmaceutically acceptable carrier, diluent and/or excipient.

21. A method of obtaining a scanning image, comprising:
i) administering to a human, humanoid, or nonhuman the suspension according to claim 14, and subsequently
ii) generating a scanning image of the human, humanoid, or nonhuman.

22. The method of claim 21, wherein the scanning image is a tomographic image.

23. A method for treating a subject comprising:
i) administering to the subject a diagnostic composition or scanning composition, comprising the particle according to claim 1, wherein the particle is capable of at least in part disturbing a magnetic field;
ii) obtaining a scanning image of the subject;
iii) determining a distribution of the particle within the subject;
iv) administering to the subject a therapeutic composition comprising the particle.

24. A method for treating a subject comprising:
i) administering to the subject a diagnostic composition or scanning composition, comprising the particle according to claim 12, wherein the particle is capable of at least in part disturbing a magnetic field;
ii) obtaining a scanning image of the subject;
iii) determining a distribution of the particle within the subject;
iv) administering to the subject a therapeutic composition comprising the particle.

25. The method according to claim 23, wherein the particle in the therapeutic composition has a higher amount of activity per particle than the particle in the diagnostic composition or scanning composition.

26. The spherical particle according to claim 1 capable of at least in part disturbing a magnetic field in a treatment of a tumour in a subject, wherein a dosage of the particle is derived from a scanning image obtained with a scanning suspension comprising particles capable of at least in part disturbing a magnetic field with the same chemical structure as the particle, based on a distribution of the particles of the scanning suspension with the same chemical structure within the subject.

27. The spherical particle according to claim 26, wherein the scanning image is obtained with tomographic imaging.

28. The spherical particle according to claim 26, wherein the scanning suspension is a therapeutic suspension comprising a spherical particle comprising lanthanide hydroxide.

29. The spherical particle according to claim 26, wherein the particle exhibits a higher amount of radioactivity per particle than the particles used for obtaining the scanning image.

30. (canceled)

31. The method of claim 22, wherein the tomographic image is generated with at least one selected from the group consisting of CLI, CT, dual energy CT, MRI, PET and SPECT.

32. The method of claim 22, wherein the tomographic image is generated with dual energy CT.

33. The spherical particle according to claim 27, wherein the scanning image is obtained with tomographic imaging generated with at least one selected from the group consisting of CLI, CT, dual energy CT, MRI, PET and SPECT.

34. The spherical particle according to claim 33, wherein the scanning image is obtained with tomographic imaging generated with dual energy CT.
